(19) United States
(12) Patent Application Publication
(10) Pub. No.: US 2008/0125729 A1
(43) Pub. Date: May 29, 2008

(54) Dressing for the Treatment of Chronic Wounds

(75) Inventor: Georg Gradl, Borgerende (DE)

Correspondence Address: LERNER GREENBERG STEMER LLP, P O BOX 2480, HOLLYWOOD, FL, 33022-2480, US

(73) Assignee: UNIVERSITAT ROSTOCK, Rostock (DE)

(21) Appl. No.: 11/935,034

(22) Filed: Nov. 5, 2007

Related U.S. Application Data
(63) Continuation of application No. PCT/DE2006/000786, filed on May 2, 2006.

(30) Foreign Application Priority Data
May 3, 2005 (DE) DE102005021292.1

(51) Int. Cl. A61F 15/00; A61M 35/00; A61F 13/56; A61M 39/00

(52) U.S. Cl. 604/313; 602/43; 604/289; 602/53

(57) ABSTRACT

A dressing for treating wounds, especially chronic wounds, includes a liquid-absorbing wound pad. A reservoir for receiving liquid is located on the side of the wound pad facing away from the body. The wound pad is connected to the reservoir via valves and/or a semi-permeable membrane so as to create a directed liquid flow from the wound pad into the reservoir.

The invention relates to a wound dressing in accordance with the preamble of claim 1.

Chronic wounds are, for example, a very common complication in geriatric patients, particularly in old people's homes and care homes and therefore constitute a medical challenge. The accumulation of wound secretion material (e.g., blood, pus, formation of pustules, and other wound fluids) promotes the growth of bacteria and encloses organisms in a scab that cause infection and retard the healing process. Such wounds may in principle, however, occur in other disease scenarios as well that are accompanied by poor supply of blood to the skin, as in the case of diabetes, for example.

Oftentimes it is desired to allow a wound to heal in a slightly moist or sealing condition, since this accelerates the healing process. However, excess wound secretion material must be removed. If this is not done, wound secretion material remains on the wound and can accumulate beneath the wound dressing, which not only is unattractive but also may result in the dressing becoming unhermetic, with the consequence that the wound is no longer covered over in a sterile manner.

A wound dressing for chronic wounds, then, should protect the wound against contamination and soiling from the outside, and at the same time the configuration should support an environment favorable to wound healing.

Over the course of time, a very great number of different wound dressings have been developed specifically for chronic wounds.

Thus, for example, DE 44 00 713 A1 describes a plaster having a wound pad which acts as an absorbent cushion to accommodate body fluid. The wound pad is used not only to accommodate body fluids but also to create a germ barrier, for thermal insulation, and for padding. In one specific embodiment it is possible, as and when required, to take off or change the wound pad without detaching the dressing.

One development by the company Hartmann has been a special wound dressing (TenderWet) which comprises a multilayer wound contact material in pad form that includes as its central constituent a superabsorbent absorbing and rinsing element. Prior to application, the wound contact material is activated with Ringer's solution which is delivered continuously to the wound. At the same time, wound exudate is accommodated in the absorbent contact material which functions as the absorption element. A disadvantage, however, is the relatively low accommodation capacity of the wound pads for body fluid, which necessitates a frequent change of dressing.

Similarly successful is treatment with osmotic substances, such as sodium chlorides and glucose, for example. In the case of this method, dressings are impregnated with an osmotic substance and applied to the wound, and then a joint dressing is mounted. The osmotic substance on wounds prevents the formation of a scab and keeps the wound soft and deformable. Moreover, the formation of epithelium at the wound is facilitated, and the wound's secretion is accommodated by the dressing.

For the treatment more particularly of extensive wounds, the vacuum therapy has become established.

In the case of vacuum therapy, the wound is covered with a special sponge and then taped over completely. Then, via a small hole, a vacuum is generated that protects the wound from infections. The edges of the wound contract, and wound secretions that are produced are taken off continuously by absorption. An advantage of this method is the promotion of blood supply and hence of healing of the wound.

This form of therapy, however, is involved and is possible only in hospitals with daily physician monitoring. Consequently the increased period of treatment also gives rise to higher treatment costs. Without this monitoring, there is a danger of the wound drying out, an event which is deleterious for the formation of epithelium and for wound healing. Furthermore, an airtight and secretion-tight closure is usually difficult to achieve.

The disadvantages of the stated possibilities for the treatment of chronic wounds lie in the fact that it necessitates several changes of dressing each day, causes the edges of the wound to pull, and is difficult to implement outside of hospitals and care establishments. This makes this highly effective and otherwise inexpensive form of wound therapy more expensive.

The problem addressed by the invention, therefore, is that of providing a wound dressing which keeps a wound in a moist condition and at the same time allows the efficient removal of the secreted wound fluid over a time period of a number of days.

This problem is solved by the provision of a wound dressing having the features of claim 1.

The wound dressing of the invention for treating wounds, more particularly chronic wounds, comprises a fluid-absorbing wound pad, a reservoir for fluid accommodation being located on the non-body-facing side of the wound pad. Wound pad and reservoir are connected via valves and/or a semipermeable membrane, which produce a directed flow of fluid from the wound pad into the reservoir. With the aid of a fastening means, the wound dressing of the invention comprising wound pad and reservoir is mounted on the skin.

The use of a reservoir on the one hand offers easy observation of the fluid status and hence of the accommodation capacity. On the other hand it is possible easily to detach and replace the reservoir without having to remove the dressing itself from the wound. By virtue of its relatively high volume, a reservoir separate from the wound pad allows the accommodation of wound fluid over several days, more particularly over a time period of 3 to 4 days. As a result of this it is possible to avoid a daily change of dressing, thus leading to cost savings. Furthermore, the use of the wound dressing of the invention allows outpatient treatment and hence increased patient mobility.

The reservoir for fluid accommodation is of cylindrical, cuboid, cubic, prismatic or ellipsoidal design. Preferably the reservoir is composed of a polymeric material, more particularly polypropylenes or polyurethanes, which is color-neutral, odor-neutral, and skin-compatible.

It is also advantageous for the reservoir to possess connections (drain) for application of a vacuum. As a result of an applied vacuum—which, however, by virtue of the construction of the wound dressing, does not act directly on the wound—the diversion of fluid from the wound pad is boosted. The wound fluid that collects in the reservoir may also be removed, in a further embodiment, by means of a syringe. Diverting the fluid produces an acceleration in the healing process.

The valves disposed between wound pad and reservoir are preferably designed as nonreturn valves. The construction of the nonreturn valves in this case is such that an elastic membrane having at least one self-sealing aperture covers the outlet aperture. The aperture is brought into a pass position by the flowing fluid. In the case of non-flowing fluid the aperture is brought automatically into a blocking position. Accordingly, the nonreturn valves produce a directed flow of wound fluid from the wound pad into the reservoir, but at the same time prevent an unwanted backflow.

Furthermore, there may be a semipermeable membrane disposed between wound pad and reservoir.

In one preferred embodiment the wound dressing is disposed on the fastening means by a fixing means. The fixing means preferably has an accommodation region which partly or fully surrounds the wound dressing comprising wound pad and reservoir. The section of the fixing means that is not used to accommodate wound pad and reservoir is connected to the fastening means.

The dimensions of the accommodation region of the fixing means correspond advantageously to the dimensions of wound pad and reservoir, with the consequence that the entire surface area of the non-body-facing side of the wound dressing is surrounded.

The fixing means is preferably composed of a hydrophobic, readily deformable material, more particularly of polymer films and/or textile materials, which on the side facing the wound dressing and the fastening means is provided with an adhesive material, more particularly polyacrylates, polyurethanes and/or epoxy resins. The adhesive produces a firm and strong bond of the fixing means and hence of the wound dressing to the fastening means.

In a further advantageous embodiment the outer edge of the fixing means is provided with incisions, which allows the wound dressing to be mounted on a rounded substrate such as a heel.

The fixing means advantageously produces a bond of the wound pad to the fastening means so hermetic that the escape of fluid from the wound pad at the edges is avoided. In principle it is necessary to provide effective sealing of the wound pad—which in certain circumstances is highly liquid-producing—in the direction of the periphery of the fastening means, since otherwise the wound fluid would cause softening of the fastening material.

The body-facing side of the wound pad of the wound dressing of the invention advantageously covers the wound completely. The pad is of cylindrical, cuboid, cubic, prismatic or ellipsoidal design.

The wound pad is composed of a liquid-accommodating absorbent material. Preferred constituents are natural, synthetic and/or chemically modified polymers, such as polyvinyl alcohol, polyurethane, polypropylene, polyesters and/or polyamides.

With advantage the wound pad can be compressed in a manner similar to a sponge. External compression of the wound pad produces a flow of fluid from the wound pad into the reservoir through the nonreturn valves. The patient is able to compress the wound pad from the outside, manually or by means of a shift in weight, as, for example, when the wound dressing is used on the back.

The wound pad is preferably impregnated with an osmotic material, such as NaCl solution and/or glucose solution. The osmotic substances are delivered to the wound over a relatively long time period, prevent the scabbing of the wound, and keep the wound soft and deformable.

The fastening means for mounting the wound dressing on the skin preferably comprises a strip of extensible and/or nonextensible material, more particularly polymeric and/or textile materials. At opposite sides, the fastening means has two area parts with a skin-friendly adhesive. When the wound dressing is applied, the area parts with the adhesive are located outside the wound region and, before the placement of the wound dressing onto the wound, are lined with a detachable protective film.

It is particularly advantageous if, in the region between the area parts furnished with the skin-friendly adhesive, the fastening means has passages that are pervious to secretions of blood and/or of exudate, and to air.

The skin-friendly adhesive applied to the fastening means preferably comprises polyacrylates and/or synthetic rubber.

Further features and advantages of the wound dressing of the invention will become apparent in the context of the description, below, of two exemplary embodiments.

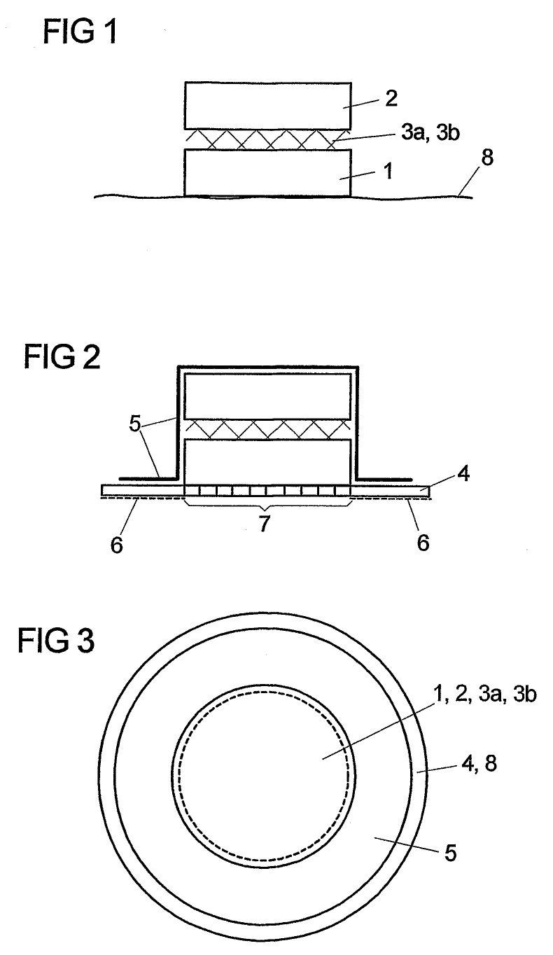

FIG. 1 shows a front elevation of a first embodiment of the wound dressing of the invention; and

FIG. 2 shows a longitudinal section of a second preferred embodiment of the wound dressing of the invention; and

FIG. 3 shows a plan view of one geometric embodiment of the wound dressing.

FIG. 1 shows a wound dressing on a surface of a body 10, comprising a wound pad 1, a reservoir 2 mounted on its non-body-facing side, and nonreturn valves 3a disposed between wound pad 1 and reservoir 2. The size of the wound pad 1 should always be selected so as to achieve full coverage of the wound by the body-facing side of the wound. The non-return valves 3a are disposed in such a way as to allow a directed flow of fluid through them, from the wound pad 1 into the reservoir 2.

FIG. 2 shows an embodiment of the wound dressing of the invention in which wound pad 1, reservoir 2, and nonreturn valves 3a are connected to a fastening means 4 for fastening on the skin. The fastening means 4 has two opposing area parts, which, when the wound dressing is applied, are located outside of the wound region, and are provided with a layer of skin-friendly adhesive 8. The region of the fastening means between these two area parts is provided with passages 9, so that the wound fluid can pass through the fastening means into the wound pad.

A fixing means 5 which connects the wound pad 1 and fastening means 4 prevents the liquid being pressed through the wound dressing. Partly or wholly, the wound dressing is surrounded by an accommodation region of the fixing means 5, the dimensions of the accommodation region matching those of wound pad 1 and reservoir 2. The section of the fixing means 5 that is not used for accommodating wound pad 1 and reservoir 2 is connected to the fastening means 4.

FIG. 3 shows a preferred geometric embodiment of the wound dressing of the invention from above. The wound dressing, comprising the wound pad 1, valves 3a or semipermeable membrane 3b, and the reservoir 2 has a circular form and is fully covered by the fixing means 5. The fixing means 5 is likewise of circular design. The accommodation region of the fixing means 5 for accommodating the wound dressing fully surrounds the wound dressing. The section of the fixing means 5 that is not part of the accommodation region is used for fixing the wound dressing on the fastening means 4 or on the body surface 8.

In terms of its embodiment the invention is not restricted to the exemplary embodiments preferred above. Instead there are a number of conceivable variants which make use of the wound consumption of the invention.

### LIST OF REFERENCE NUMERALS

1 wound pad
2 reservoir
3a nonreturn valves
3b semipermeable membrane
4 fastening means
5 fixing means
6 skin-friendly adhesive
7 region of the fastening means with passages
8 body surface

1-26. (canceled)

27. A wound dressing for treating wounds, comprising:
a fluid-absorbing wound pad having a body-facing side and an opposite, non-body-facing side;
a reservoir for fluid accommodation disposed on said non-body-facing side of said wound pad; and
flow means selected from the group consisting of valves and/or a semi-permeable membrane disposed to establish communication between said wound pad and said reservoir for producing a directed flow of fluid from said wound pad into said reservoir.

28. The wound dressing according to claim 27, which comprises fastening means for mounting said wound pad and said reservoir on the skin of a patient.

29. The wound dressing according to claim 27, wherein said reservoir has a shape selected from the group consisting of cylindrical, cuboid, cubic, prismatic, and ellipsoid.

30. The wound dressing according to claim 27, wherein the reservoir has connections for application of a vacuum in order to boost the diversion of fluid from the wound pad.

31. The wound dressing according to claim 27, wherein said reservoir includes connections for application of syringes.

32. The wound dressing according to claim 27, wherein said reservoir is formed of polymeric material.

33. The wound dressing according to claim 32, wherein said reservoir is formed of at least one polymeric material selected from the group consisting of polypropylenes and polyurethanes.

34. The wound dressing according to claim 27, wherein said flow means are formed of non-return check valves disposed between said wound pad and said reservoir.

35. The wound dressing according to claim 34, wherein said check valves comprise an elastic membrane covering an outlet aperture and having at least one self-sealing aperture, wherein said aperture is configured to be brought into a pass position for the fluid, by way of a flowing fluid, and to be automatically brought, in the case of nonflowing fluid, into a blocking position for the fluid.

36. The wound dressing according to claim 27, wherein said flow means are formed of a semi-permeable membrane disposed between said wound pad and said reservoir.

37. The wound dressing according to claim 28, which comprises a fixing means connecting said wound pad and said reservoir with said fastening means.

38. The wound dressing according to claim 37, wherein said fixing means is formed with an accommodation region for said wound pad and said reservoir.

39. The wound dressing according to claim 37, wherein said accommodation region of said fixing means is formed to partly or fully surround said wound pad and said reservoir.

40. The wound dressing according to claim 37, wherein said fixing means includes a section that is not used to accommodate said wound pad and said reservoir, and said section is connected to said fastening means and/or attachable to the skin of a patient.

41. The wound dressing according to claim 37, wherein said fixing means is composed of a hydrophobic material and said fixing means, on a side thereof facing said wound pad, said reservoir, said fastening means, and/or the skin of the patient, is provided with an adhesive material.

42. The wound dressing according to claim 27, wherein said wound pad is formed with a shape selected from the group consisting of cylindrical, cuboid, cubic, prismatic, or ellipsoid.

43. The wound dressing according to claim 27, wherein an area of the body-facing side of said wound pad corresponds at least to an area of a wound to be covered.

44. The wound dressing according to claim 27, wherein said wound pad is compressible.

45. The wound dressing according to claim 27, wherein said wound pad is formed with fluid-accommodating absorbent material.

46. The wound dressing according to claim 27, wherein said wound pad includes polymers selected from the group consisting of natural polymers, synthetic polymers, and chemically modified polymers as constituents.

47. The wound dressing according to claim 45, wherein said wound pad contains polyvinyl alcohol, polyurethane, polypropylene, polyesters, and/or polyamides.

48. The wound dressing according to claim 27, wherein said wound pad is impregnated with an osmotically active material.

49. The wound dressing according to claim 47, wherein said wound pad is impregnated with a solution selected from the group consisting of an NaCl solution and a glucose solution.

50. The wound dressing according to claim 28, wherein said fastening means is a strip of extensible and/or nonextensible material.

51. The wound dressing according to claim 28, wherein said fastening means includes, on opposite sides thereof, two area parts with a skin-friendly adhesive, and wherein said area parts, when the wound dressing is applied, are located outside a wound region, and said area parts are lined with a detachable, protective film configured with removal prior to a placement of the wound dressing on the wound.

52. The wound dressing according to claim 51, wherein said fastening means includes, in a region between said area parts, skin-friendly adhesive and passages that are pervious to body fluids and to air.

53. The wound dressing according to claim 51, wherein said skin-friendly adhesive comprises polyacrylates and/or synthetic rubber.

54. The wound dressing according to claim 27 configured for dressing chronic wounds.
